(19) United States
(12) Patent Application Publication
(10) Pub. No.: US 2004/0223990 A1
(43) Pub. Date: Nov. 11, 2004

(54) Cosmetic compositions for caring for and/or making up the skin, lips and/or integuments

(75) Inventors: Jean Mondet, Aulnay Sous Bois (FR); Philippe Ilekti, Paris (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/838,329

(22) Filed: May 5, 2004

Related U.S. Application Data
(60) Provisional application No. 60/472,752, filed on May 23, 2003.

(30) Foreign Application Priority Data
May 5, 2003 (FR) 03 05447

(51) Int. Cl. A61K 7/00

(52) U.S. Cl. 424/401

(57) ABSTRACT

A cosmetic composition for caring for and/or making up the skin, the lips and/or the integuments includes, in a physiologically acceptable medium containing at least one fatty phase and at least an effective amount of exfoliated phyllosilicates derived from at least one phyllosilicate intercalated with one or more molecules of a non-polyphenolic intercalating agent.

### CROSS-REFERENCE TO RELATED APPLICATIONS

This application claims the benefit of French Application No. 03 05447 filed on May 5, 2003 and U.S. Provisional Application No. 60/472,752 filed on May 23, 2003, the entire disclosures of which are incorporated by reference herein.

### BACKGROUND OF THE INVENTION

The present invention relates to cosmetic compositions for caring for and/or making up the skin, lips and/or integuments.

More particularly, the compositions according to the invention may constitute makeup products for the skin, lips and/or integuments. Preferably, the compositions according to the invention have non-therapeutic care and/or treating properties.

The present invention is directed to improving the persistence of a cosmetic effect of a cosmetic composition, especially for making up the skin, including, but not limited to, the eyelids, the lips and the integuments (e.g., eyelashes, eyebrows and nails). The cosmetic products include, but are not limited to, lipsticks, mascaras, eyeliners, foundations, powders, makeup rouges, eyeshadows, nail varnish, products for nail care and body makeup products.

Conventionally, makeup compositions are applied to the surface to be made up in the form of a deposit intended to afford the expected coloring and/or sheen and/or matting effects.

By definition, a matting product is a product that unifies the complexion. Makeup or skincare compositions endowed with matting properties are generally used to solve the sheen problems arising from an excess of sebum and to improve the long-term staying power of the makeup, which has a tendency to visually degrade in the course of the day. These matting compositions generally give the skin a matt appearance resulting from their ability to scatter light at the surface of the skin. They are also advantageous for attenuating skin defects such as microreliefs, wrinkles, fine lines, pores or color variations.

Coloring and sheen effects are aesthetic effects more particularly sought for lipsticks, mascaras and eyeliners. As regards the matting effect, this more particularly concerns skincare or skin makeup compositions.

For obvious reasons, it is desirable for the effects mentioned above to last for as long as possible. For example, for lipsticks, mascaras and eyeliners, it is important to obtain a prolonged duration of the coloration and/or sheen. For foundations, powders, makeup rouges, eyeshadows and body makeup products, it is important to obtain a colored and matt effect that is persistent and long-lasting, despite types of attack such as rubbing and/or secretions of sebum or sweat to which the applied coat of makeup is subject.

To do this, cosmetic compositions and especially makeup compositions contain, besides a fatty phase such as wax and/or oil, at least one filler, which is generally a mineral filler. It is thus known practice to introduce fillers into cosmetic compositions in order to adjust their properties in terms of texture and especially to improve their properties in terms of staying power. The mineral or organic fillers conventionally used are, for example, silica, nylon powders or bentone, which is a phyllosilicate material in which the outer surfaces of its stacks of lamellae have been treated so as to make them organophilic. As regards matting compositions, they generally contain powders that absorb sebum and the excess oil of the composition that is not absorbed by the skin. Among the matting powders of natural or synthetic origin that may especially be mentioned are some of the above fillers, for instance talc, silica and nylon powders, but also starch, mica, polyethylene powders and polymethyl (meth)acrylate powders.

In point of fact, the size of these conventional fillers, which is generally on the micron scale, and/or their intrinsic matt nature may significantly affect the aesthetic properties of cosmetic compositions in which they are incorporated.

Thus, in the case of certain cosmetic compositions, for instance lipsticks, it is necessary to use a large amount of filler in order to sufficiently limit the migration of the oils in the fine lines of the skin and in particular the lips and/or in order to obtain good staying power. The matt nature of these fillers may thus, under these conditions, cause a significant loss of sheen of the corresponding cosmetic composition.

Similarly, in the case of matting compositions, a powdery appearance is very often noted, which is a consequence of an excessively large size of the said fillers, which is incompatible with a natural effect.

### SUMMARY OF THE INVENTION

The inventors have found, unexpectedly, that it is possible to overcome these drawbacks while at the same time providing cosmetic suitable compositions having desirable properties, such as long-lasting staying power.

In the present case, the inventors have discovered that exfoliated phyllosilicates are advantageous additives for obtaining cosmetic compositions having particularly useful properties. Among such properties achieved in various embodiments of the invention may be mentioned good texture, hold, glossiness, mattness and absence of migration. One or more of these and other desirable properties can be obtained through use of such exfoliated phyllosilicates.

Phyllosilicates, which are more commonly known as clays, generally denote silicates in lamellar form.

The term “intercalated phyllosilicates” refers to phyllosilicates that have been treated with organic or mineral compounds in order to introduce molecules of these compounds into the interfoliar spaces of the phyllosilicates, firstly to increase the distance between the sheets, and secondly to give them an organophilic nature. Beneficially, a sufficient amount of molecules may be adsorbed between two adjacent phyllosilicate lamellae so as to increase their interfoliar space to a size of, for example, at least 5 angströms, and in particular of about 10 angströms, to promote the consecutive exfoliation of the intercalated material in the form of separate sheets. This exfoliation, also known as delamination, is generally performed under shear in the presence of an organic solvent or in a polymer matrix to obtain “exfoliated phyllosilicates”.

At the current time, two exemplary intercalation techniques are preferred.

The first exemplary technique, which is described especially in WO 93/04118, includes exchanging hydrophilic mineral cations, originally present between the phyllosilicate lamellae, with organic “onium” cations, generally quaternary alkyl-ammoniums. The phyllosilicates thus intercalated are used in their exfoliated form in polymer matrices in order especially to reinforce their mechanical properties. Quaternary ammonium cations are in particular well known for converting highly hydrophilic phyllosilicates such as calcium or sodium montmorillonites into organophilic phyllosilicates.

The second exemplary technique involves modifying the nature of the ligands to which the cations of the interfoliar spaces are coordinated. In the natural state, the cations of the interfoliar spaces are coordinated to water molecules. This second method involves replacing these water molecules with specific organic hydrocarbon-based molecules comprising at least one polar group. For example, U.S. Pat. No. 5,721,306 proposes, as an intercalating agent, hydrocarbon-based molecules containing at least one polar group of hydroxyl, carbonyl, carboxyl, amine, amide, ether, ester, and even also sulfate, sulfonate, sulfinate, sulfamate, phosphate, phosphonate or phosphinate type, or an aromatic group, capable of interacting with the metal cations bound to the surfaces of the phyllosilicate lamellae. These are especially alkylpyrrolidone, polyvinylpyrrolidone, polyvinyl alcohol and polyoxyalkylenes, polyamide or polyimide derivatives. The phyllosilicates thus intercalated are subsequently exfoliated. The disclosed phyllosilicates in U.S. Pat. No. 5,721,306 are used as thickeners for preparing formulations of a viscous thixotropic gel type, and in particular as carriers for a wide variety of active materials. More recently, U.S. Pat. No. 6,500,411 discloses the use of phyllosilicates intercalated and exfoliated using natural polyphenol derivatives, such as lignin, in aqueous cosmetic compositions especially of antisun formulation type.

In various exemplary embodiments, the present invention is directed to cosmetic compositions for caring for and/or making up the skin, lips and/or integuments, comprising a physiologically acceptable medium containing at least one fatty phase and at least an effective amount of exfoliated phyllosilicates derived from at least one intercalated phyllosilicate with one or more non-polyphenolic intercalating agent.

As used herein, the term “deriving” in the expression “exfoliated phyllosilicates deriving from at least one intercalated phyllosilicate” can refer to obtaining of phyllosilicates in a form of sheets obtained from intercalated phyllosilicates following the application of an efficient shear stress allowing at least partial or total exfoliation. The nature of the shear stress liable to be applied is further detailed bellow.

In various exemplary embodiments, the physiologically acceptable medium comprises at least one liquid fatty phase.

In various exemplary embodiments, the composition is an emulsion.

In exemplary embodiments, the present invention is directed to cosmetic compositions for caring for and/or making the skin, the lips and/or integuments in a form of emulsion, the compositions comprising, in a physiologically acceptable medium comprising at least one fatty phase, at least an efficient amount of at least one intercalated phyllosilicate with one or more intercalating agent, and being free of any emulsifying or co-emulsifying agent.

Advantageously, emulsions containing exemplary exfoliated phyllosilicates in accordance with the invention may comprise a smaller amount of emulsifying and co-emulsifying agents, and may even be totally free from those agents.

The inventors have thus discovered that exemplary phyllosilicates in accordance with the invention can be advantageously used as stabilizing agents to enhance the stability of cosmetic emulsions, which ordinarily require an efficient amount of surfactants or emulsifying and co-emulsifying agents. Exemplary phyllosilicates allow significant, and possibly complete, reductions of the presence of classic surfactants and emulsifying and co-emulsifying agents usually required, providing an obvious advantage with respect to innocuousness.

Various exemplary embodiments of the compositions according to the invention may be advantageously transparent or translucent and/or capable of giving a transparent or translucent coat.

Within the meaning of the invention, “bulk” transparency or translucency can mean that a layer of the composition with a given thickness allows a portion of the visible light to pass. If this portion of the visible light is scattered, the composition can be referred to as a bulk translucent composition and if, on the other hand, visible light is not scattered, then the composition can be referred to as a bulk transparent composition.

Various exemplary embodiments of the present invention are more specifically directed towards financially upgrading intercalated and then exfoliated phyllosilicates in specific cosmetic compositions.

According to another exemplary embodiment, the present invention is also directed to the use of exfoliated phyllosilicates in accordance with the invention as agents for adjusting the staying power and especially the migration-resistance and transfer-resistance properties of a cosmetic composition for making up and/or caring for the skin, the lips and/or the integuments.

A further subject of the present invention, according to another exemplary embodiment, is the use of exfoliated phyllosilicates in accordance with the invention for stabilizing emulsions, in particular aqueous-phase-in-oil phase or oil-phase-in-aqueous-phase emulsions.

Another subject of the present invention, according to another of its exemplary embodiments, is the use of exfoliated phyllosilicates in accordance with the invention as agents for texturizing the liquid fatty phase of a cosmetic composition for caring for and/or making up the skin, lips and/or integuments.

In exemplary embodiments of the present invention, exfoliated phyllosilicates lack any carrier activity with regard to any active substance that may be present in the cosmetic composition. In other words, it is possible that exemplary phyllosilicates need not be employed in the packaging of an active substance into the cosmetic composition.

Various exemplary embodiments of the present invention include processes for making up and/or caring for the skin, the lips, the nails and/or the integuments, comprising the application to the skin, the lips, the nails and/or the integuments of at least one composition in accordance with the present invention.

In various exemplary embodiments, the invention also includes synthetic supports, such as false eyelashes, on which at least one coat of a composition according to the invention is present on all or a part of its surface.

### DETAILED DESCRIPTION OF EXEMPLARY EMBODIMENTS

Exfoliated Phyllosilicates

As specified previously, exfoliated phyllosilicates deriving from at least one intercalated phyllosilicate may be present in exemplary cosmetic compositions according to the invention in various degrees of exfoliation.

Thus, some phyllosilicates may be present in a completely exfoliated form, that is to say in the form of a single sheet, and others, on the other hand, in a partially exfoliated form, that is to say in a form comprising two or more sheets still associated. Advantageously, at least 50%, notably 70%, in particular 90%, indeed even 95% of exemplary exfoliated phyllosilicates may comprise less than 10 sheets, in particular less than 5 sheets, particularly less than 3 sheets, indeed even they may comprise only one sheet of phyllosilicate.

All of these forms may, of course, coexist in the exemplary cosmetic compositions according to the invention, indeed even mixed with non-exfoliated intercalated phyllosilicates.

Exemplary exfoliated phyllosilicates according to the invention may be characterized by a shape factor, which is the ratio of the largest dimension to the smallest dimension. This shape factor may range from 50 to 2000, especially from 75 to 1500, and in particular from 200 to 1000.

Exemplary exfoliated phyllosilicates according to the invention may have a mean thickness of more than 5 Å and a maximum thickness of less than 100 Å, in particular ranging from 10 Å to 50 Å.

Given their small thicknesses, which are on the scale of a few angströms, exemplary exfoliated phyllosilicates advantageously do not provide any opacity, in contrast to conventional fillers. Specifically, like conventional matting compositions, exemplary compositions according to the invention and especially those intended to be applied to the skin, for instance foundations, are advantageously found to be effective for unifying the complexion and especially for attenuating skin imperfections, while at the same time giving the made-up skin a natural appearance. Such compositions have good surface light-scattering power and are stable over time.

Exemplary phyllosilicates moreover give a cosmetic composition advantageous properties in terms of staying power without, however, adversely affecting its aesthetic qualities such as the color effect, the sheen or the matt nature.

Likewise, exemplary phyllosilicates can be used to maintain stabilization of emulsions and, as such, may be used efficiently for totally or partially substitute and/or replace surfactants generally required to maintain such stabilization.

Exemplary phyllosilicates that may be used according to the invention are more particularly derived from the intercalation of mineral clays of smectite type, for instance montmorillonites, especially sodium, potassium and/or calcium montmorillonites, nontronites, beidellites, volkonskoites, hectorites, saponites, sauconites, sobockites, stevensites, svinfordites and vermiculites, and mixtures thereof. Phyllosilicates of montmorillonite type are particularly suitable for the invention.

Exemplary exfoliated phyllosilicates according to the invention have advantageously, adsorbed onto the surface of their lamellae, at least one intercalating agent. The intercalating agent can be intercalated between the sheets of the exemplary intercalated phyllosilicates which are used to prepare compositions according to the invention.

Exemplary exfoliated phyllosilicates contain at least 15% by weight, especially at least 20% by weight and more particularly at least 30% by weight of intercalating agent relative to the weight of dry phyllosilicate (comprising less than 5% water).

As specified above, exemplary exfoliated phyllosilicates according to the invention are free of polyphenolic intercalating agent.

For the purposes of the present invention, exemplary agents that can be especially excluded under this term are natural intercalating agents, comprising at least one phenolic unit, for instance lignin, lignosulfonates, humates, tannates, and salts and derivatives thereof.

Intercalating agents may be adsorbed onto all or a part of the surface of the phyllosilicate lamellae and may be especially present with phyllosilicate or cations present at its surface by hydrogen, ionic or covalent type and/or hydrophilic or Van der Waals interactions.

In general, exemplary intercalating agents according to the invention have in their chemical structure at least one hydrophobic group, especially a C₄ to C₅₀ alkyl, C₄ to C₅₀ alkylene and/or C₄ to C₅₀ alkylaryl chain.

In the context of the present invention, exemplary intercalating agents most particularly considered are compounds chosen from:

oniums containing at least one C₁-C₅₀ and especially C₄-C₅₀ hydrocarbon-based chain, and

intercalating agents “containing a polar group”, for instance organic compounds of polymeric or non-polymeric nature, containing at least one aromatic ring or at least one polar group chosen from carbonyl, hydroxyl, polyol (including glycol, glycerol, etc.), carboxylic acid, aldehyde, ketone, amine, amide (linear or cyclic, in particular pyrrolidone or caprolactam), ester, lactone, ether, or even sulfate, sulfonate, sulfinate, sulfamate, phosphate, phosphonate and phosphinate groups.

As mentioned above, the intercalating agents of the onium type can partially or totally replace the hydrophilic mineral ions (Na⁺, K⁺, etc.) present in phyllosilicates in the natural state. This is also referred to as ion-exchange intercalation. As regards the intercalating agents containing at least one polar group, they can partially or totally replace the water molecules that initially coordinated the hydrophilic metal cations present between the lamellae.

Intercalating Agents of Onium Type

The term “oniums” can refer to ammonium, sulfonium or phosphonium groups as indicated, for example, in WO 93/04118 assigned to Allied Signal.

Among these oniums that may be mentioned more particularly are primary ammoniums N⁺H₃R₁, secondary ammoniums, tertiary ammoniums and quaternary ammoniums, especially of the type N⁺H₂R₁R₂, N⁺HR₁R₂R₃ and N⁺R₁R₂R₃R₄ in which the groups R₁, R₂, R₃ and R₄, which may be identical or different, represent C₁-C₅₀ hydrocarbon-based chains, at least one of which is more particularly of C₄-C₅₀, these hydrocarbon-based chains possibly being linear, branched or cyclic and saturated or unsaturated, and possibly comprising one or more hetero atoms such as O, S, N, Si or P. They may especially be C₄ to C₅₀ alkyl, C₄ to C₅₀ alkylene or C₄ to C₅₀ alkylaryl chains.

In exemplary embodiments of the present invention, one of the substituents R₁ to R₄ of the omnium group can be aromatic species (benzyl or phenyl) or arylalkyl, then the other group being alkyl chain C₄ to C₅₀ as defined above.

As non-limiting illustrations of intercalating agents of onium type that may be used according to the invention, mention may be made especially of those described in patent application WO 93/04118 assigned to Allied Signal, the dionium or multionium intercalating agents described in EP 1 038 834 and WO 00/09605 assigned to Amcol and the intercalating agents containing an onium group, preferably primary, secondary or tertiary ammoniums containing two alkyl chains of C≧10, as described in JP 04 357 108 assigned to Nippon Paint.

Among these oniums, the ones that are most particularly suitable are primary, secondary, tertiary or quaternary ammoniums, preferably quaternary ammoniums, containing at least one C₄ to C₁₀ alkyl chain, this chain preferably being linear or branched and preferably saturated, for instance a butyl, isobutyl, pentyl, isopentyl, hexyl, heptyl, 2-ethylhexyl, octyl, nonyl, decyl, undecyl, dodecyl, octadecyl, etc. chain. Among these ammonium cations, those containing at least one alkyl chain of dodecyl or octadecyl type are most particularly suitable.

Intercalating Agents Containing a Polar Group

Among the intercalating agents containing a polar group, distinction will be made between (a) those that are not of polymeric nature and (b) those that are of polymeric nature.

As used herein, the term “compound of polymeric nature” can refer to a compound containing at least two repeating units, especially at least three repeating units, in particular at least ten repeating units or even at least fifteen repeating units. It may moreover be composed of a single repeating unit (homopolymer) or of at least two repeating units of different nature (copolymer).

(a) Intercalating Agents Containing a Polar Group of Non-Polymeric Nature

Exemplary agents comprise at least one polar group as defined above and, preferably, at least one hydrophobic chain, especially a C₄-C₅₀ hydrocarbon-based chain, which may be linear, branched or cyclic and saturated or unsaturated, and may moreover contain hetero atoms such as O, S, N, Si or P. It may especially be a C₄-C₅₀ alkyl, C₄-C₅₀ alkylene or C₄-C₅₀ alkylaryl chain. Such compounds are described especially in U.S. Pat. No. 5,721,306, EP 780 340 and U.S. Pat. No. 6,242,500.

As illustrations of compounds of this type, mention may be made of those comprising as polar group at least one group chosen from C₆-₂₄ alcohol groups, glycerols containing at least one C₆-C₂₄ chain, C₆-C₂₄ carboxylic acids, especially those described in EP 780 340, amide groups, preferably cyclic amides like lactams, such as pyrrolidone or caprolactam derivatives, and substituted with an aromatic group, for instance those described in U.S. Pat. No. 6,242,500, or with a C₄-C₅₀ alkyl, preferably C₈-C₃₀ and preferentially C₁₂-C₂₅ alkyl chain, for instance alkylpyrrolidones with a C₄ to C₅₀ and in particular a C₈ to C₃₀ alkyl chain. It may in particular be dodecylpyrrolidone, described in particular in Beall, G. W., “Nanocomposites produces utilizing a novel dipole clay surface modification in polymer-clay composites,” Chemistry and Technology of Polymer Additives (1999), 266-280, Editor: Al-Malaika, Sahar; Publisher: Blackwell, Oxford UK.

(b) Intercalating Agents Containing a Polar Group, of Polymeric Nature

Exemplary agents are more particularly synthetic oligomers or homo- or copolymers comprising at least one aromatic nucleus or a polar group as defined above. The MW may range from 300 to 200,000 and in particular from 500 to 40,000. Such agents are described, for example, in U.S. Pat. No. 5,837,763.

Exemplary oligomers or polymers may be hydrophilic or hydrophobic.

Illustrations of these hydrophilic polymeric intercalating agents that may especially be mentioned include polyvinylpyrrolidone (PVP) derivatives, polyvinyl alcohol (PVA) derivatives, especially when they are virtually in their hydrolysed polyvinylacetate form, in other words containing less than 5% of residual acetyl groups, polyacrylic derivatives in their polymeric and copolymeric form and more particularly in the form of their metal salts, polymethacrylic acid (PMAA) derivatives, polyvinyloxazolidone (PVO) derivatives, polyvinylmethyloxazolidone (PVMO) derivatives and polyvinyloxazoline derivatives.

Other hydrophilic polymeric intercalating agents that may also be mentioned include copolymers of the units mentioned above, copolymers between these same units or copolymers with other hydrophilic or strongly polar monomers, such as: hydroxyethyl (meth)acrylate, 2-hydroxypropyl (meth)acrylate, methyl (meth)acrylate, vinyl acetate, (meth)acrylamide, N,N-dimethylacrylamide, crotonic acid, maleic anhydride and methyl vinyl ether.

Exemplary polymeric intercalating agents may also be organophilic or even lipophilic, provided that they contain at least one or more polar group(s) as defined above. Copolymers obtained by reaction between a polar and hydrophilic monomer as chosen above (vinylpyrrolidone, vinyloxazoline, vinyloxazolidone, vinyl alcohol or (meth)acrylic acid) with at least one monomer that is more organophilic or even lipophilic, such as (meth)acrylic acid esters such as ethyl, butyl, isobutyl, tert-butyl, hexyl, cyclohexyl, octyl, 2-ethylhexyl, nonyl, decyl, undecyl, dodecyl, octadecyl or behenyl (meth)acrylates, vinyl esters such as propionate, versatate or benzoate; (meth)acrylamides such as diacetoneacrylamide, butyl(meth)acrylamide, tert-butyl(meth)acrylamide, tert-hexyl(meth)acrylamide and tert-octyl(meth)acrylamide; olefins such as ethylene, propylene, butene, isobutene, hexene, octene, dodecene, octadecene, eicosene, styrene and substituted styrenes, can be selected in particular.

Other water-soluble or polyhydric alcohols and polymeric polyols such as polysaccharides are also capable of constituting polymeric intercalating agents.

Certain phyllosilicate/intercalating agent combinations such as montmorillonite-PVP, montmorillonite-PVA and montmorillonite-alkylpyrrolidone, for instance montmorillonite-dodecylpyrrolidone, and mixtures thereof, are particularly advantageous for the scope of the invention.

The intercalation of exemplary phyllosilicates in the context of the present invention may be performed according to conventional protocols such as those described, for example, in U.S. Pat. No. 5,721,306 and WO 93/04118.

In exemplary embodiments, intercalating agents may be introduced or adsorbed into the interfoliar spaces of the phyllosilicate according to the following embodiment: the intercalation is performed by intimately mixing the phyllosilicates by extrusion or stirring with an impeller that preferably produces high shear, so as to form an intercalating composition comprising the phyllosilicate in an intercalating polymer, an aqueous solution of intercalating agent or an organic solution of intercalating agent. In order to obtain sufficient intercalation for the purpose of exfoliation, the intercalating agent is generally placed in contact with the phyllosilicate in the intercalating composition in an intercalating agent/phyllosilicate weight ratio of at least about 1/20, especially of at least about 1/10, more particularly from about ½ to ⅕ or even about ¼, so as to obtain efficient intercalation of the agent between the adjacent phyllosilicate lamellae. The interfoliar space may thus be increased from 10 to 100 angströms to ensure consecutive, easy and total exfoliation. The intercalating vehicle, preferably water, where appropriate mixed with an organic solvent, may be introduced after pre-dissolution or -dispersion of the intercalating agent in the vehicle, or directly mixed with the dry intercalating agent and the dry phyllosilicate. The amount of intercalating agent is generally at least 15% by weight, especially at least 20% by weight and more particularly at least 30% by weight relative to the weight of dry phyllosilicate (comprising less than 5% of water). This amount may range especially from 20% to 50% by weight relative to the weight of the phyllosilicate in dry form.

Exemplary polymeric intercalating agents may often be obtained by direct polymerization (homo or copolymerization) of monomers which were previously intercalated between the sheets of phyllosilicates.

As regards the exfoliation of the phyllosilicates thus intercalated, it can be performed conventionally, generally by applying to the medium in which the intercalated phyllosilicates are dispersed a shear rate that is sufficient to produce the desired delamination. This aspect is more particularly developed hereinbelow.

There are, incidentally, a certain number of intercalated and exfoliated phyllosilicates that are already commercially available.

As illustrations of exfoliated phyllosilicates that are suitable for the invention, mention may be made more particularly of those sold by:

the company Nanocor in the United States. As examples of clays (montmorillonite type) intercalated with a quaternary alkylammonium, i.e. via ion exchange, mention may be made of Nanomer 1.24 T, 1.30 TC and 1.34 TCN; as examples of clays intercalated with an organic pyrrolidone derivative (ion-dipole technology via exchange of the coordinated water), mention may be made of Nanomer 1.35 K and 1.46 D, which are also described in: “Advances in monomer additives for clay/polymer nanocomposites” LAN, T. (NANOCOR), Additives 99, International Conference, 8th, San Francisco, March 22-24, 1999 (1999) Paper 12/1—Paper 12/11, Publisher=Executive Conference Management, Plymouth, Mic.; and

the company Southern Clay Products in the United States, which also produces clays intercalated with a quaternary alkylammonium, e.g., Cloisite 25A and Cloisite 30B, which are cited in the article by Poittevin, B., Polymer 43, 4017-23 (2002).

Exemplary exfoliated phyllosilicates according to the invention can be present in an amount that is effective to give the cosmetic composition good properties in terms of staying power and especially transfer resistance and/or migration resistance, or alternatively to gel the fatty phase and/or the organic phase of the said composition.

In particular, exfoliated phyllosilicates may be present in a proportion of from 0.05% to 20% by weight, especially from 0.1 to 15% by weight and more particularly from 0.5% to 10% by weight in the cosmetic composition.

To the extent exfoliated clays are used for the purpose of stabilizing an emulsion, an efficient amount is, evidently, liable to significantly vary depending on whether or not those clays are associated with other surfactants. Adjustments are with the skill of the ordinary artisan.

Phyllosilicates can be introduced directly in exfoliated form into the composition. However, the invention also includes compositions in which the exfoliated phyllosilicates are generated in situ, for example by simple stirring of the cosmetic composition containing intercalated phyllosilicates, i.e. in a non-exfoliated form, or indeed even shaking of the system into which it is packaged. As specified previously, exfoliated phyllosilicates according to the invention may be present as a mixture with non-exfoliated intercalated phyllosilicates of identical or different chemical nature.

Physiologically Acceptable Medium

The term “physiologically acceptable medium” can refer to a non-toxic medium that may be applied to human skin or lips. The physiologically acceptable medium is generally suited to the nature of the support onto which the composition is to be applied and also the aspect in which the composition is intended to be packaged.

Fatty Phase

In exemplary embodiments, compositions, especially when they are intended to be applied to the lips or the skin, for example in the form of a foundation, may especially comprise at least one fatty substance that is liquid at room temperature (25° C.) and at atmospheric pressure and/or a fatty substance that is solid at room temperature and atmospheric pressure, such as waxes, pasty fatty substances and gums, and mixtures thereof. The fatty phase may also contain oil-gelling and oil-structuring agents of organic nature and/or lipophilic organic solvents.

Exemplary embodiments of the compositions according to the invention can comprise at least one liquid fatty phase. In particular, the fatty phase can constitute the continuous phase or the dispersed phase of an emulsion and in particular of a microemulsion.

Compositions can have, for example, a continuous fatty phase which can comprise less than 10% by weight of water, in particular less than 5% by weight of water, indeed even less than 1% by weight of water with respect to its total weight and in particular can be in the anhydrous form.

According to various exemplary embodiments of the invention, compositions contain less than 70% by weight, especially less than 50% by weight and in particular less than 20% by weight of an aqueous phase, relative to the total weight of the composition. As used herein, the term “aqueous phase” denotes a phase consisting of one or more hydrophilic solvent(s) such as water, glycols, la glycerol or alcohols, and mixtures thereof.

In particular, the liquid fatty phase may constitute the continuous phase or the dispersed phase of an emulsion.

The term “structured liquid fatty phase” can refer to a stiffened or gelled liquid fatty phase or only thickened.

The term “gelled or thickened liquid fatty phase” is understood to mean that the viscosity of this fatty phase is increased because of its combination with an exemplary exfoliated phyllosilicate according to the invention.

In exemplary embodiments, the fatty phase of the composition according to the invention may especially comprise, as liquid fatty substance, at least one volatile or non-volatile oil, or a mixture thereof.

In exemplary embodiments, the oily phase of the composition according to the invention may be present in a proportion varying from 1 to 80%, in particular from 1 to 50% by weight relative to the total weight of the composition.

As used herein, the term “volatile oil” can refer to any oil capable of evaporating on contact with the skin in less than one hour, at room temperature and atmospheric pressure. Volatile oils of the invention can be volatile cosmetic oils, which are liquid at room temperature, having a non-zero vapour pressure, at room temperature and atmospheric pressure, ranging in particular from 0.01 to 300 mmHg (1.33 Pa to 40 000 Pa) and preferably greater than 0.3 mmHg (30 Pa).

The term “non-volatile oil” can refer to an oil that remains on the skin at room temperature and atmospheric pressure for at least several hours and that especially has a vapour pressure of less than 0.01 mmHg (1.33 Pa).

These volatile or non-volatile oils may be hydrocarbon-based oils, especially of animal or plant origin, silicone oils or mixtures thereof. The term “hydrocarbon-based oil” means an oil mainly containing hydrogen and carbon atoms and possibly oxygen, nitrogen, sulfur and/or phosphorus atoms.

Volatile hydrocarbon-based oils may be chosen from hydrocarbon-based oils containing from 8 to 16 carbon atoms, and especially branched C₈-C₁₆ alkanes, for instance C₈-C]₆ isoalkanes of petroleum origin (also known as isoparaffins), for instance isododecane (also known as 2,2,4,4,6-pentamethylheptane), isodecane, isohexadecane, and for example the oils sold under the trade names Isopars® or Permetyls®, branched C₈-C₁₆ esters such as isohexyl neopentanoate, and mixtures thereof. Other volatile hydrocarbon-based oils, for instance petroleum distillates, especially those sold under the name Shell Solt® by the company Shell, may also be used.

Volatile oils that may also be used include volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile oils may be present in compositions according to the invention in a content ranging from 0.1% to 98% by weight, especially from 1% to 65% by weight, and in particular from 2% to 50% by weight, relative to the total weight of the composition.

Non-volatile oils may be chosen especially from non-volatile fluoro and/or silicone hydrocarbon-based oils.

Non-volatile hydrocarbon-based oils that may especially be mentioned include:

hydrocarbon-based oils of animal origin,

hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, maize oil, apricot oil, castor oil, shea oil, avocado oil, olive oil, soybean oil, sweet almond oil, palm oil, cottonseed oil, hazelnut oil, macadamia oil, jojoba oil, alfalfa oil, poppyseed oil, pumpkin oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stéarineries Dubois or those sold under the names Miglyol 810®, 812® and 818® by the company Dynamit Nobel,

synthetic ethers containing from 10 to 40 carbon atoms,

linear or branched hydrocarbons of mineral or synthetic origin, such as petroleum jelly, polydecenes, hydrogenated polyisobutene such as parleam, and squalane, and mixtures thereof,

synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance propylene glycol dioctanoate; hydroxylated esters, for instance isostearyl lactate or diisostearyl malate; polyol esters and pentaerythritol esters,

fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,

higher fatty acids such as oleic acid, linoleic acid or linolenic acid, and

silicone oils of polymethylsiloxane (PDMS) type, and mixtures thereof.

More specifically, non-volatile silicone oils that may be used in the composition according to the invention may be non-volatile polydimethylsiloxanes (PDMS), polydimethylsiloxanes comprising alkyl or alkoxy groups, which are pendent and/or at the end of a silicone chain, these groups each contain from 2 to 24 carbon atoms, phenylsilicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyltrimethylsiloxysilicates.

Non-volatile oils may be present in compositions according to the invention in a content ranging from 0.01% to 90% by weight, especially from 0.1% to 85% by weight and in particular from 1% to 70% by weight relative to the total weight of the composition.

More generally, fatty substances that are liquid at room temperature and atmospheric pressure may be present in a proportion of from 0.01% to 90% by weight and especially from 0.1% to 85% by weight relative to the weight of the fatty phase.

As regards fatty substances that are solid at room temperature and atmospheric pressure, they may be chosen from waxes, pasty fatty substances and gums, and mixtures thereof. Such solid fatty substances may be present in a proportion of from 0.01% to 50%, especially from 0.1% to 40% and in particular from 0.2% to 30% by weight relative to the total weight of the fatty phase.

Thus, in exemplary embodiments, compositions according to the invention may comprise at least one fatty substance that is pasty at room temperature.

The term “pasty” can refer to a lipophilic, fatty compound with a reversible solid/liquid change of state exhibiting in the solid state, an anisotropic crystalline arrangement, and comprising, at a temperature of 23° C., a liquid fraction and a solid fraction.

The term “pasty compound” can refer to a compound having a hardness, at 23° C., ranging from 0.001 to 0.5 MPa, in particular from 0.002 to 0.4 MPa.

Hardness of a sample can be measured by a method of penetrating a probe into a sample of compound and in particular using a texture analyzer (for example, TA-XT2i from Rhéo) equipped with a stainless steel cylinder of with diameter 2 mm. The hardness measurement is carried out at 20° C. at the center of 5 samples. The cylinder is introduced into each sample at a pre-rate of 1 mm/s and then at a measuring rate of 0.1 mm/s, the total displacement being 0.3 mm. The recorded hardness value is that of the maximum peak observed.

Further, at a temperature of 23° C., a pasty compound can be in the form of a liquid fraction and a solid fraction. In other words, the starting melting temperature of the pasty compound is less than 23° C. The liquid fraction of the pasty compound measured at 23° C., can represent 9 to 97% by weight relative to the total weight of the compound. This liquid fraction at 23° C. can represent, in particular, from 15 to 85%, particularly from 40 to 85% by weight relative to the total weight of the compound.

Liquid fraction by weight of the pasty compound at 23° C. is equal to the ratio of the enthalpy of fusion consumed at 23° C. to the enthalpy of fusion of the pasty compound.

Enthalpy of fusion of pasty compounds is the enthalphy consumed by the compound to change from the solid state to the liquid state. The pasty compound is in “solid state” when the whole of its mass is in a cristalline solid form. The pasty compound is in “liquid state” when the whole of its mass is in a liquid form.

Enthalpy of fusion of a pasty compound is equal to the area under the curve of the termogram obtained using a differential scanning calorimeter (D.S.C). Such as the NDSC 2920 calorimeter sold by TA Instrument, with rise in temperature of 5 or 10° C. to minute, according to the ISO standard 11357-3:1999. Enthalpy of fusion of a pasty compound is the amount of energy required to change the compound from the solid state to the liquid state. It is expressed in J/g.

Enthalpy of fusion consumed at 23° C. is the amount of energy absorbed by the sample to change from the solid state to the state which it exhibits at 23° C., composed of a liquid fraction and a solid fraction.

In exemplary embodiments, the liquid fraction of the pasty compound measured at 32° C. represents, in particular, from 30 to 100% by weight of the compound, particularly from 80 to 100%, more particularly from 90 to 100% by weight of the compound. To the extent the liquid fraction of the pasty compound measured at 32° C. is equal to 100%, the temperature of the end of the melting range of the pasty compound is less than or equal to 32° C.

In exemplary embodiments, the liquid fraction of the pasty compound measured at 32° C. is equal to the ratio of the enthalpy of fusion consumed at 32° C. to the enthalpy of fusion pasty compound. The enthalpy of fusion consumed at 32° C. is computed in the same way as the enthalpy of fusion consumed at 23° C.

In exemplary embodiments, the fatty substances can be hydrocarbon-based compounds optionally of polymeric type; they may also be chosen from silicone compounds; they may also be in the form of a mixture of hydrocarbon-based and/or silicone compounds. In the case of a mixture of different pasty fatty substances, the hydrocarbon-based pasty compounds (mainly containing carbon and hydrogen atoms and optionally ester groups) are preferably used in a majority proportion.

Among the pasty compounds that may be used in compositions according to the invention, mention may be made of lanolins and lanolin derivatives, for instance acetylated lanolins, oxypropylenated lanolins or isopropyl lanolate, and mixtures thereof. Esters of fatty acids or of fatty alcohols, especially those containing 20 to 65 carbon atoms may also be used, for instance triisostearyl citrate or cetyl citrate; arachidyl propionate; polyvinyl laurate; cholesterol esters, for instance triglycerides of plant origin such as hydrogenated plant oils, viscous polyesters, and mixtures thereof. Triglycerides of plant origin that may be used include hydrogenated castor oil derivatives, such as “Thixinr®” from Rheox.

Mention may also be made of silicone pasty fatty substances such as polydimethylsiloxanes (PDMS) of high molecular weight and in particular those with pendent chains of the alkyl or alkoxy type containing from 8 to 24 carbon atoms, and a melting point of 20-55° C., for instance stearyldimethicones, especially those sold by the company Dow Coming under the trade names DC2503® and DC25514®, and mixtures thereof.

Pasty fatty substances may be present in compositions according to the invention in a content ranging from 0.01% to 50% by weight, preferably ranging from 0.1% to 45% by weight and better still ranging from 0.2% to 30% by weight relative to the total weight of the composition.

In exemplary embodiments, compositions according to the invention may also comprise a wax. Waxes may be solid at room temperature (25° C.), with a reversible solid/liquid change of state, having a melting point of greater than 30° C. which may be up to 200° C., a hardness of greater than 0.5 MPa and having an anisotropic crystalline organization in the solid state. They may be a hydrocarbon-based wax, a fluoro wax and/or a silicone wax and may be of animal, plant, mineral or synthetic origin. It may be chosen, for example, from beeswax, camauba wax, candelilla wax, paraffin waxes, hydrogenated castor oil, silicone waxes or microcrystalline waxes, and mixtures thereof.

In particular, waxes may be present in the form of a wax-in-water emulsions.

Waxes may be present in compositions according to the invention in a content ranging from 0.01% to 50% by weight, in particular from 0.1% to 30% by weight and especially from 0.2% to 20% by weight relative to the total weight of the composition.

Emulsion

As used herein, the term “emulsion” can refer to a system of two immiscible liquids of which one is finely divided in droplets into the other. The dispersed phase can be referred to as an “internal or discontinuous phase”. The dispersing phase can be referred to as a “continuous or external phase”. Emulsions in which the dispersed phase is lipophilic, such as vegetal or mineral oil, and the dispersing phase is hydrophilic, such as water, can be referred to as “aqueous emulsions” (O/W: oil in water). Emulsions in which the dispersed phase is hydrophilic and the dispersing phase is lipophilic can be referred to as “oily emulsions” (W/O: water in oil). Multiple emulsions are also known, such as W/O/W: water in oil in water.

Thus, exemplary emulsions comprise a lipophilic and a hydrophilic phase, this latter being not necessarily water.

In particular, exemplary compositions in the form of emulsions may be transparent or translucent and/or may be capable of giving a transparent or translucent coat.

In exemplary embodiments, compositions comprise at least one emulsifying agent and, if appropriate, at least one co-emulsifying agent in an amount lower than 30%, in particular lower than 20%, particularly lower than 10%. According to another embodiment, compositions comprise at least one emulsifying agent and, if appropriate, at least one co-emulsifying agent in an amount ranging from 0.2 to 30% by weight relative to the total weight of the composition, in particular from 0.3 to 20% by weight and advantageously from 0.5 to 15% by weight relative to the total weight of the composition.

In exemplary embodiments, compositions comprise less than 0.5% by weight relative to the total weight of the composition of emulsifying and co-emulsifying agent.

In exemplary embodiments, compositions according to the invention in the form of emulsions are advantageously free from any emulsifying agent allowing its stabilization, to the exception of the exfoliated phyllosilicate in accordance with the invention.

A skilled artisan readily knows how to select, for compositions comprising selected aqueous phase and fatty phase, in a selected ratio, the emulsifying agent and possibly the co-emulsifying agent from the prior art and their respective proportions for obtaining a stable emulsion. The term “stable emulsion” as used herein refers to an emulsion which, when placed in a transparent container, itself placed in an incubator at 45° C. for two months, does not undergo a separation of phases (or exudate) at the end of this period. The separation of phases (or exudation) is visually detected through the transparent wall of the container, when it is withdrawn from the intubator.

Exemplary compositions may, notably be in the form of emulsions comprising an aqueous phase and an oily phase, dispersed one in another, for example in the form of emulsions water-in-oil (W/O) or oil-in-water (O/W) or multiple emulsions (W/O/W or O/W/O) or in the form of emulsions chosen among the usual emulsions or particular emulsions such as:

emulsions O/W comprising oily globules comprising a lamellar liquid crystal coating, such as described in EP-A-641 557 and EP-A-705 593;

emulsions O/W without emulsifying agent, stabilized with hydrodispersible anionic polymer, such as desribed in EP-A- 864 320;

emulsions O/W comprising polymers derived from sulfonic 2-acrylamido-2-methylpropane acid (AMPS polymer), such as described in EP-A-815 844;

emulsions O/W stabilized with hydrophobic AMPS polymers, such as described in EP-A-1 069 142, WO-A-2002/43689, WO-A-2002/44231, WO-A-2002/44271, WO-A-2002/44270, WO-A-2002/43686, WO-A-2002/44267, WO-A-2002/43688, WO-A-2002/43677, WO-A-2002/43687, WO-A-2002/44230;

fluid emulsions comprising thermo associative polymers, such as described in EP-A-1 355 990, EP-A-1 355 625, EP-A-1 307 501, EP-A-1 363 954;

emulsions O/W obtained according to the PIT process (emulsion obtained with phase inversions, PIT: phase inversion temperature), such as described in WO-A-89/11907, DE-A-431 8171, and EP-A-815 846; and

nanoemulsions such as described in the EP-A-728 460, EP-A-780 114, EP-A-780 115, EP-A-879 789, EP-A-1 010 413, EP-A-1 010 414, EP-A-1 010 415, EP-A-1 010 416, EP-A-1 013 338, EP-A-1 016 453, EP-A-1 018 363, EP-A-1 020 219, EP-A-1 025 898, EP-A-1 120 102, EP-A-1 120 101, EP-A-1 160 005, EP-A-1 172 077 and EP-A-1 353 629.

In exemplary embodiments, the proportion of oily phase in emulsion may range from 1 to 80% by weight, and in particular from 1 to 50% by weight relative to the total weight of the composition. The oils, the emulsifying and co-emulsifying agents possibly present, used in compositions to form emulsions are chosen among those which are usually used in the cosmetic or dermatologic field. The emulsifying agent and co-emulsifying agent when present, are generally in a proportion ranging from 0.2 to 30% by weight, in particular from 0.3 to 20% by weight, and more particularly from 0.5 to 15% by weight relative to the total weight of the composition. Further, the emulsion may comprise lipidic vesicles.

Emulsifying agents may be chosen from the group consisting of amphoteric, anionic, cationic or non ionic emulsifying agents, alone or in combination. Emulsifying agents are chosen in an appropriate way according to the continuous phase of the emulsion to be obtained (W/O or O/W). To the extent the emulsion is in multiple form, it usually comprises an emulsifying agent in the primary emulsion and an emulsifying agent in the external phase into which is introduced the primary emulsion.

Exemplary emulsifying agents, which can be used in the preparation of W/O emulsion, of sorbitane or glycerol or glyucid ether or alkylester; the silicone-sufactants such as dimethicone copolyol such as the mixture of dimethicone copolyol and cyclomethicones sold as DC 5225C and DC 3225C by Dow Corning, and as alkyldimethicone copolyol such as Lauryl methicone copolyol sold as “Dow Corning 5200 formulation Aid” by Dow Corning, cetyl dimethicone copolyol sold as “aAbil EM 90” by Goldschmidt and the mixture of polyglyceryl-4 isostearate/cetyl dimethicone copolyol/hexyl laurate sold as “Abil WE09®” by Goldschmidt. One or more co-emulsifying agent(s) may be added which may be advantageously chosen in the group consisting of polyols branched chain fatty acid esters, and in particular sorbitan and/or glycerol branched chain fatty acid esters, as for example, polyglyceryl isostearate, such as the product Isolan G134 commercialized by Goldschmidt, sorbitan isostearate, such as the product ARLACEL 987 commercialized by ICI, sorbitan and glycerol isostearate, such as the product ARLACEL 986 commercialized by ICI, and their mixtures.

Exemplary emulsifying agents, which may be used in the preparation of O/W emulsions, include non ionic emulsifying agents such as oxyalkylenated polyol fatty acid esters (in particular polyoxyethylenated) and for example the glycol polyethylene stearate such as PEG-100 stearate, PEG-50 stearate and PEG-40 stearate; and their mixtures such as the mixtures of glyceryl monostearate and glycol polyethylene stearate (100 OE) commercialized as SIMULSOL 165 by SEPPIC; oxylalkylenatede sorbitan fatty acid ester comprising for example from 20 to 100 OE, and for example those commercialized as TWEEN 20 or TWEEN 60 from UBIQEMA; the oxyalkylenatede fatty alcohol ether (oxyethylenated and/or oxypropylenated); alkoxylated or not glucid ester, as sucrose stearate and as sesqui stearatge methylglucose PEG 20; sorbitan esters such as sorbitan palmitate commercialized as SPAN 40 by UBIQEMA; fatty alcohol diacid esters, such as dimyristyltartrate; the mixtures of those emulsifying agents such as for example the mixture of glycerylstearate and PEG-100 stearate, commercialized as ARLACEL 165 by UNIQEMA; and the mixtures comprising emulsifying agents, such as the mixtures comprising dimyristyltartrate, cetearylic alcohol, Pareth-7, and PEG-25 laureth-25, sold as COSMACOL PSE from SASOL (named as CTFA: dimyrilstyltartrate/cetearyl alcohol/12-15 Pareth7/PPG 25 laureth 25).

In exemplary embodiments, co-emulsifying agents may be added to the emulsifying agents, such as for example fatty alcohols in C₈ to C₂₆, as for example cetylic alcohol, stearylic alcohol and their mixtures (cetearylic alcohol), dodecanolopctyl, butyloctanol-2, hexyldecanol-2, undecylpentadecanole-2 or oleic alcohol, or fatty acid.

Emulsions may also be prepared without any emulsifying surfactant or comprising less than 0.5% of such agent by weight relative to the total weight of the composition, by using appropriate compounds, such as for example polymers having emulsifying properties such as polymers commercialized as CARBOPOL 1342 and PEMULEN from Noveon; or polymers in emulsion such as SEPIGEL 305 from SEPPIC (INCI: polyacrylamide/C₁₃-C₁₄ isoparaffine/laureth-7); particles of ionic or non ionic polymers, more particularly particles of anionic polymers such as, in particular, isophtalic acid polymers or sulfoisophtalic acid, and in particular phtalate/sulfoisophtalate/glycol (for example diethylene glycol)/phtalate/isophtalate/1,4-cyclohexane-dimethanol copolymer, (INCI: diglycol/CHDM/isophtalate/SIP copolymer) sold as EASTMAN AQ POLYMER (AQ 35S, AQ 38S, AQ 55S, AQ 48 ULTRA) from Eastman Chemical. Emulsions without emulsifying agents may be also prepared which are stabilized with silicone particles or metal oxide particles such as TiO₂ or else.

Film-Forming Polymers

Exemplary compositions according to the invention may also comprise at least one film-forming polymer.

As used herein, the term “film-forming polymer” can refer to a polymer capable of forming, by itself or in the presence of an auxiliary film-forming agent, a continuous film on a support.

Film-forming polymers may be organic or inorganic. In one particular embodiment of the invention, the organic film-forming polymer is at least one polymer chosen from the group comprising:

liposoluble film-forming polymers, and

lipodispersible film-forming polymers in the form of non-aqueous dispersions of polymer particles, preferably dispersions of polymer particles, where appropriate surface-stabilized with at least one stabilizer, in one or more silicone and/or hydrocarbon-based oils; these non-aqueous dispersions are also known as “NADs”.

I. Liposoluble Polymers

A. Liposoluble polymers may be of any chemical nature and especially include: amorphous liposoluble homopolymers and copolymers of olefins, of cycloolefins, of butadiene, of isoprene, of styrene, of vinyl ethers, esters or amides or of (meth)acrylic acid esters or amides comprising a linear, branched or cyclic C₄₋₅₀ alkyl group, and preferably amorphous.

Liposoluble copolymers that may be mentioned include:

(i) acrylic-silicone-grafted polymers containing a silicone skeleton and acrylic grafts or containing an acrylic skeleton and silicone grafts, such as the product sold under the name SA 70.5 by 3M and those described in U.S. Pat. No. 5,725,882, U.S. Pat. No. 5,209,924, U.S. Pat. No. 4,972,037, U.S. Pat. No. 4,981,903, U.S. Pat. No. 4,981,902, U.S. Pat. No. 5,468,477, U.S. Pat. No. 5,219,560 and EP 0 388 582.

(ii) liposoluble polymers bearing fluoro groups belonging to one of the classes described above, in particular those described in U.S. Pat. No. 5,948,393, and the alkyl (meth)acrylate/perfluoroalkyl (meth)acrylate copolymers described in EP 0 815 836 and U.S. Pat. No. 5,849,318.

(iii) polymers or copolymers resulting from the polymerization or copolymerization of an ethylenic monomer, comprising one or more ethylenic units, which are preferably conjugated (or dienes). As polymers or copolymers resulting from the polymerization or copolymerization of an ethylenic monomer, it is possible to use vinyl, acrylic or methacrylic copolymers, which may be block copolymers, such as diblock or triblock copolymers, or even multiblock copolymers of various forms. The film-forming agent, comprising at least one ethylenic unit, may comprise, for example, a styrene (S) block, an alkylstyrene (AS) block, an ethylene/butylene (EB) block, an ethylene/propylene (EP) block, a butadiene (B) block, an isoprene (I) block, an acrylate (A) block, a methacrylate (MA) block or a combination of these blocks.

(iv) copolymers of N-vinylpyrrolidone and of olefins, the number of carbon atoms of which olefins is >8, e.g. N-vinylpyrrolidone/hexadecene or N-vinyl-pyrrolidone/eicosene copolymer.

(v) homopolymers or copolymers of liposoluble vinyl esters, for instance polyvinyl laurates, polyvinyl stearate and copolymers thereof with vinyl acetate.

(vi) homopolymers or copolymers of (meth)acrylic esters or amides. The methacrylic ester monomers then result from the esterification of (meth)acrylic acid with an alcohol containing a number of carbon atoms >4 and preferably ≧8, for example 2-ethylhexyl (meth)acrylate, n-octyl (meth)acrylate, dodecyl (meth)acrylate, stearyl (meth)acrylate or behenyl (meth)acrylate. These long-chain (meth)acrylates may be copolymerized with other (meth)acrylic esters, vinyl esters or styrene.

B. liposoluble polycondensates, especially not comprising groups that give hydrogen bonds, in particular polyesters containing C₄-50 alkyl side chains, polyesters resulting from the condensation of dimeric fatty acids, or polyesters comprising a silicone segment in the form of a block, graft or terminal group, which are solid at room temperature.

C. liposoluble and amorphous polysaccharides comprising alkyl (ether or ester) side chains, in particular alkylcelluloses bearing linear or branched, saturated or unsaturated C₁ to C₈ alkyl radicals, such as ethylcellulose and propylcellulose.

In general, film-forming liposoluble polymers that may be used according to the invention may have a molecular weight of between 1000 and 500,000 and preferably between 2000 and 250,000, and a glass transition temperature of between −100° C. and +300° C., especially between −50° C. and +100° C. and in particular between −10° C. and +90° C.

II. Lipodispersible Polymers: Non-Aqueous Dispersions (NAD) of Polymer Particles

These are stable non-aqueous dispersions of polymer particles, which are generally spherical, of one or more polymers, in a physiologically acceptable liquid fatty phase, such as hydrocarbon-based oils or silicone oils. These dispersions are generally known as non-aqueous dispersions (NAD) of polymers. These dispersions may be in particular in the form of stable dispersions of polymer nanoparticles in the said fatty phase. According to one particular variant, the size of these nanoparticles ranges between 5 nm and 600 nm. However, it is possible to obtain polymer particles ranging up to 1 μm.

Polymer dispersions of this type advantageously offer the possibility of varying the glass transition temperature (Tg) of the polymer or of the polymer system (polymer plus additive of plasticizer type) and thus of changing from a hard polymer to a more or less soft polymer. It is thus possible to adjust the mechanical properties of the composition as a function of the intended application, for example as regards the deposited film.

Polymers in dispersion that may be used in compositions according to the invention preferably have a molecular weight from about 2000 to 10 000 000 and a Tg from −100° C. to 300° C., in particular from −50° C. to 50° C. and especially from −10° C. to 100° C.

It is possible to use film-forming polymers that preferably have a low Tg, less than or equal to the temperature of the skin and in particular less than or equal to 40° C. The dispersion thus obtained can form a film when it is applied to a support.

Among the film-forming polymers that may be mentioned are, most particularly, vinyl or acrylic homopolymers or copolymers of free-radical type, especially having a Tg of less than or equal to 40° C. and more particularly ranging from −10° C. to 30° C., used alone or as a mixture.

The expression “polymer of free-radical type” can refer to a polymer obtained by polymerization of monomers containing an unsaturation, more particularly ethylenic unsaturation; each monomer being capable of homopolymerization (unlike polycondensates). These polymers of free-radical type may be more particularly vinyl polymers or copolymers, in particular acrylic polymers.

Polymers of free-radical type that are preferably used are copolymers of (meth)acrylic acid and of an alkyl (meth)acrylate, more particularly of a C₁-C₄ alkyl. Preferably, methyl acrylates may be used, optionally copolymerized with an acrylic acid.

In a non-limiting manner, exemplary polymers in dispersion according to the invention may be chosen from the following polymers or copolymers: polyurethanes, polyurethane-acrylics, polyureas, polyurethane-polyureas, polyurethane-polyesters, polyurethane-polyethers, polyesters, polyesteramides, fatty-chain polyesters, vinyl and/or acrylic polymers or copolymers, silicone/acrylic copolymers, polyacrylamides, silicone polymers, for example silicone polyurethanes or silicone-acrylics, and fluoropolymers, and mixtures thereof.

Polymer(s) in oily dispersion can include (as active material or solid) from 0.1% to 60% by weight of the composition, especially from 2% to 40% and better still from 4% to 25%. In the case of the presence of a stabilizer that is solid at room temperature, the content of solid in dispersion represents the total number of polymer and of stabilizer.

Liposoluble or lipodispersible polymers in exemplary compositions according to the invention may also be used in an amount ranging from 0.01% to 40% relative to the total weight of the composition, especially from 1% to 20%, for instance from 1% to 10%.

Film-forming polymers may be combined with auxiliary film-forming agents. Such film-forming agents can include any compound known to those skilled in the art as being capable of satisfying the desired function, and may be chosen especially from plasticizers and coalescers.

Particulate Phase

Exemplary embodiments of compositions according to the invention may also comprise an additional particulate phase, which may be present in a proportion of from 0.01% to 40% by weight, especially from 0.01% to 30% by weight and in particular from 0.05% to 20% by weight relative to the total weight of the composition.

They may especially comprise additional pigments and/or nacres and/or fillers conventionally used in cosmetic compositions.

The term “pigments” can refer to white or colored, mineral or organic particles that are insoluble in the liquid hydrophilic phase, which are intended to color and/or opacify the composition. The term “fillers” should be understood as meaning colorless or white, mineral or synthetic, lamellar or non-lamellar particles. The term “nacres” should be understood as meaning iridescent particles produced especially by certain molluscs in their shell, or alternatively synthesized.

Pigments may be present in compositions in a proportion of from 0.01% to 25% by weight, in particular from 0.05% to 20% by weight and especially from 0.1% to 15%, and in particular from 0.5% to 10% by weight relative to the weight of the composition.

Exemplary mineral pigments that may be used in the invention include titanium oxide, zirconium oxide or cerium oxide, and also zinc oxide, iron oxide or chromium oxide, ferric blue, manganese violet, ultramarine blue and chromium hydrate. Among exemplary organic pigments that may be used in the invention, mention may be made of carbon black, pigments of D & C type, and lakes based on cochineal carmine or on barium, strontium, calcium or aluminium, or alternatively the diketopyrrolopyrroles (DPP) described in documents EP-A-542 669, EP-A-787 730, EP-A-787 731 and WO-A-96/08537. The amount and/or choice of these pigments are generally adjusted taking into account the amount of exfoliated intercalated phyllosilicates present in the cosmetic composition under consideration.

Nacres may be present in embodiments of compositions according to the invention in a proportion of from 0.01% to 25% by weight, especially from 0.01% to 15% by weight and in particular from 0.02% to 5% by weight relative to the total weight of the composition.

Nacreous pigments may be chosen from white nacreous pigments such as mica coated with titanium or with bismuth oxychloride, colored nacreous pigments such as titanium mica with iron oxides, titanium mica especially with ferric blue or with chromium oxide, titanium mica with an organic pigment of the abovementioned type and nacreous pigments based on bismuth oxychloride.

Additional fillers may be present in a proportion of from 0.01% to 40% by weight, especially from 0.01% to 30% by weight and in particular from 0.02% to 20% by weight relative to the total weight of the composition. Their amount is also generally adjusted taking into account the amount of exfoliated intercalated phyllosilicates.

Exemplary fillers include spherical fillers, for instance talc, zinc stearate, mica, kaolin, polyamide (Nylon®) (Orgasol® from Atochem) powders, polyethylene powders, tetrafluoroethylene polymer (Teflon®) powders, starch, boron nitride, polymer microspheres such as those of polyvinylidene chloride/acrylonitrile, for instance Expancel® (Nobel Industrie), acrylic acid copolymers (Polytrap® from the company Dow Corning), silicone resin microbeads (for example Tospearls® from Toshiba) and organopolysiloxane elastomers.

In exemplary embodiments, compositions may also comprise water-soluble or liposoluble dyes in a content ranging from 0.01% to 6% by weight and especially ranging from 0.01% to 3% by weight relative to the total weight of the composition. Liposoluble dyes include, for example, Sudan Red, DC Red 17, DC Green 6, β-carotene, soybean oil, Sudan Brown, DC Yellow 11, DC Violet 2, DC Orange 5 and quinoline yellow. Water-soluble dyes include, for example, beetroot juice and methylene blue.

In exemplary embodiments, compositions according to the invention may also comprise any ingredient conventionally used in the fields under consideration and more especially in cosmetics and dermatology. Exemplary ingredients include vitamins, antioxidants, trace elements, softeners, sequestering agents, fragrances, basifying or acidifying agents, preserving agents, UV-screening agents, hydrophilic or lipophilic active agents, and mixtures thereof. Amounts of these various ingredients are those conventionally used in the fields under consideration, for example from 0.01% to 20% of the total weight of the composition.

Needless to say, a person skilled in the art will take care to select this or additional compound(s), and/or the amount thereof, such that the advantageous properties of the composition according to the invention are not, or are not substantially, adversely affected by the envisaged addition.

Compositions of the invention may be obtained according to the preparation processes conventionally used in cosmetics or dermatology.

According to one preferred embodiment of the invention, clays will be used for the cosmetic formulations, preferably pre-intercalated montmorillonites, which can be exfoliated:

(a) either in a pregel containing at least one of the solvents of the final formulation. This may be, for example, a volatile or non-volatile solvent chosen from ethanol, a polyol such as propylene glycol, glycerol, a volatile aliphatic hydrocarbon such as isododecane, a polar or non-polar hydrocarbon-based oil or a volatile or non-volatile silicone; or

(b) directly in the formulation or in one of the phases of the formulation (this is, for example, an emulsion or a dispersion), i.e. in the presence of the other solvents and/or oils and/or polymers and/or pigments and fillers.

Whether it is performed according to (a) or (b), intercalated phyllosilicates can be exfoliated by any means capable of delaminating at least about 80% by weight of the said phyllosilicate. In exemplary embodiments, the shear rate required to achieve this type of exfoliation may require a shear rate of at least 10 s⁻¹, or even more. The upper limit of this shear rate is not critical.

In general, this shear rate ranges from about 10 s⁻¹ to about 20 000 s⁻¹ and more particularly from about 100 s⁻¹ to about 10 000 s⁻¹. In certain cases, it may be advantageous to combine this shear with heating and/or a pressure increase.

As more particularly concerns the shear per se, it may be achieved using various conventional devices. Shear may be achieved using mechanical means, via thermal shock, change of pressure or via ultrasonication. The choice of the mode of shear falls within the knowledge of a skilled artisan.

Shear obtained via mechanical methods, especially such as stirrers, homogenizers or dispersers of the Moritz® or Ultra-Turrax® type, a Banbury® blender, Brabender® blenders and extruders, especially of Kneader® type, are most particularly suitable for the invention.

In the particular case of a mechanical shear, especially in an extruder, the temperature of the medium to be exfoliated, the length of the extruder, the residence time of this medium in the extruder and the type of extruder selected, i.e., single-screw/twin-screw, etc., are all variables capable of controlling the shear force to be applied for exfoliation.

Exfoliation may be sufficient when it affords at least 80% by weight, especially at least 85% by weight, in particular at least about 90% by weight or even 95% by weight of exfoliated phyllosilicate.

Phyllosilicates thus formed advantageously have a thickness ranging from the thickness of individual layers to the thickness of one to five associated layers.

Exemplary compositions may be in various forms, depending on their intended use. Cosmetic compositions may thus be in any form normally used for topical application and especially in an anhydrous form, in the form of an oily or aqueous solution, an oily or aqueous gel, an oil-in-water, water-in-oil, wax-in-water or water-in-wax emulsion, a multiple emulsion, or a dispersion of oil in water by means of vesicles located at the oil/water interface, of direct or inverse emulsion

Exemplary compositions may be in the form of a cast product, in a dish or in the form of a stick, especially in the case of a lipstick or a lip care product.

Compositions may also be in various other forms, for example in the form of a more or less viscous liquid, a gel or a paste.

Compositions may also be in the form of a semi-solid or a solid, for example a cake to be moistened at the time of use so as to allow it to be taken up.

Advantageously, compositions containing an oily continuous phase can comprise less than 10% by weight of water, especially less than 5% by weight of water, and in particular that may be anhydrous.

In various exemplary embodiments, cosmetic compositions can constitute, inter alia, a lipstick, a lip balm, a liquid gloss, a lipstick paste, a blusher, a varnish nail, a lip pencil, a solid or liquid foundation, in particular a cast foundation, a product for caring for and/or making up natural or synthetic nails, a concealer, a product for “correcting” or “embellizing” the complexion, an eyeliner, a mascara, an eyeshadow, a product for making up the body or hair, or an antisun product or coloring product for the skin. In particular, it may be a nail varnish.

This invention is illustrated by the following examples, which are merely for the purpose of illustration.

### EXAMPLE 1 AND COMPARATIVE EXAMPLE

Preparation of Nail Varnish According to the Invention

A nail varnish according to the invention is prepared by formulating phyllosilicate made of tallow derived fatty amine modified montmorillonite (Nanomer I.34 TCN from Nanocor).

A nail varnish from the prior art is also prepared according to the same process, by exchanging by weigth to weight the phyllosilicate with stearyldimethylbenzylammonium chloride modified hectorite (Bentone® 27V from Elementis).

A nitrocellulosic thixotropic gel is obtained by mixing at 25° C. under appropriate stirring:

13 g of nitrocellulose (film-forming agent);

7 g of intercalated phyllosilicates or hectorite;

0.3 g of citric acid (swelling agent);

8 g of isopropylic alcohol (volatile solvent); and

butyl acetate q.s. 100 g.

100 parts of the gel thus obtained were mixed at 25° C. to 450 parts with the following non-colored, non-thixotropic base composition:

8 g of plasticizer;

24 g of a mixture of nitrocellulose and co-film forming agent;

5 g of isopropylic alcohol; and

butylacetate/ethylacetate 50/50 q.s. 100 g.

A nail varnish of the following composition is thus obtained (weight in %):

| | |
|---|---|
| film-forming agent (nitrocellulose resin) | 22% |
| plasticizer | 6.5% |
| isopropylic alcohol | 5.3% |
| phyllosicilate or hectorite | 1.3% |
| citric acid | 0.05% |
| butylacetate | 39.2% |
| ethylacetate | 26.1% |

The glossiness of this composition, is measured with a glossiness measuring device, according to the conventional manner, with the following method.

A layer having a thickness of around 300 μm is spread on a contrast card LENETA (ref. FORM 1A PENOPAC) with an automatic spreader. The layer covers at least the white bottom of the card. The coat is left to dry. The glossiness is measured at 20° and 60° on the white base with a glossiness measuring device BYK GARDNER, ref. MICRO TRI-GLOSS. A mean value for the glossiness is obtained between 0 and 100. The measured values are listed below for each of the tested compositions.

| | Transparent | Transparent varnish |
|---|---|---|
| | varnish prepared | prepared with the |
| | with Bentone ® 27 V from | phyllosilicate Nanomer I.34 |
| | Elementis | TCM from Nanocor |
| Glossy 20°/60° | 53/85 | 63/86 |

### EXAMPLE 2 AND COMPARATIVE EXAMPLE

Preparation of Nail Varnish

In a double-screw mixer, the following compounds are mixed:

20 g of hectorite or phyllosilicate as defined in Example 1;

30 g of cellulose acetobutyrate from Eastman Chemical CAB 3810.5; and

50 g of n-ethyl-o, p-toluene sulfonamide from PAN-AMERICANA (Resimpol 8).

5 parts of the above-obtained gel are mixed with 95 parts of the nitrocellulosic thixotropic gel described in Example 1.

A layer having a thickness of around 300 μm is spread on a contrast card LENETA (ref. FORM 1A PENOPAC) with an automatic spreader. The layer covers at least the white bottom of the card. The coat is left to dry. The glossiness is measured at 20° and 60° on the white base with a glossiness measuring device BYK GARDNER, ref. MICRO TRI-GLOSS. A mean value for the glossiness is obtained between 0 and 100. The measured values are listed below for each of the tested compositions.

The obtained results for each of the tested phyllosilicates are listed below.

| | | Transparent varnish |
|---|---|---|
| | Transparent varnish prepared | prepared with the |
| | with Bentone ® 27 V from | phyllosilicates Nanomer |
| | Elementis | from Nanocor I34.TCN |
| Glossy 20°/60° | 54/79 | 71/87 |

While this invention has been described in conjunction with the exemplary embodiments and examples outlined above, various alternatives, modifications, variations, improvements and/or substantial equivalents, whether known or that are or may be presently unforeseen, may become apparent to those having at least ordinary skill in the art. Accordingly, the exemplary embodiments of the invention, as set forth above, are intended to be illustrative, not limiting. Various changes may be made without departing from the spirit and scope of the invention. Therefore, the invention is intended to embrace all known or later developed alternatives, modifications, variations, improvements and/or substantial equivalents.

What is claimed is:

1. A cosmetic composition for caring for and/or making up the skin, lips and/or integuments, comprising a physiologically acceptable medium containing at least one fatty phase and at least an effective amount of exfoliated phyllosilicates, wherein the exfoliated phyllosilicates are derived from at least one phyllosilicate intercalated with one or more molecules of a non-polyphenolic intercalating agent.

2. The composition according to claim 1, wherein the exfoliated phyllosilicates have a shape factor ranging from 50 to 2000.

3. The composition according to claim 2, wherein the exfoliated phyllosilicates have a shape factor ranging from 75 to 1500.

4. The composition according to claim 3, wherein the exfoliated phyllosilicates have a shape factor ranging from 200 to 1000.

5. The composition according to claim 1, wherein at least 50% of the exfoliated phyllosilicates are formed from less than 10 lamellae.

6. The composition according to claim 5, wherein at least 70% of the exfoliated phyllosilicates are formed from less than 10 lamellae.

7. The composition according to claim 6, wherein at least 90% of the exfoliated phyllosilicates are formed from less than 10 lamellae.

8. The composition according to claim 7, wherein at least 95% of the exfoliated phyllosilicates are formed from less than 10 lamellae.

9. The composition according to claim 1, wherein at least 50% of the exfoliated phyllosilicates are formed from less than 5 lamellae.

10. The composition according to claim 9, wherein at least 70% of the exfoliated phyllosilicates are formed from less than 5 lamellae.

11. The composition according to claim 10, wherein at least 90% of the exfoliated phyllosilicates are formed from less than 5 lamellae.

12. The composition according to claim 11, wherein at least 95% of the exfoliated phyllosilicates are formed from less than 5 lamellae.

13. The composition according to claim 1, wherein at least 50% of the exfoliated phyllosilicates are formed from less than 3 lamellae.

14. The composition according to claim 13, wherein at least 70% of the exfoliated phyllosilicates are formed from less than 3 lamellae.

15. The composition according to claim 14, wherein at least 90% of the exfoliated phyllosilicates are formed from less than 3 lamellae.

16. The composition according to claim 15, wherein at least 95% of the exfoliated phyllosilicates are formed from less than 3 lamellae.

17. The composition according to claim 1, wherein at least 50% of the exfoliated phyllosilicates are formed of one phyllosilicate lamella.

18. The composition according to claim 17, wherein at least 70% of the exfoliated phyllosilicates are formed of one phyllosilicate lamella.

19. The composition according to claim 18, wherein at least 90% of the exfoliated phyllosilicates are formed of one phyllosilicate lamella.

20. The composition according to claim 19, wherein at least 95% of the exfoliated phyllosilicates are formed of one phyllosilicate lamella.

21. The composition according to claim 1, having a content of exfoliated phyllosilicates ranging from 0.05% to 20% by weight relative to the total weight of the composition.

22. The composition according to claim 21, having a content of exfoliated phyllosilicates ranging from 0.1% to 15% by weight relative to the total weight of the composition.

23. The composition according to claim 22, having a content of exfoliated phyllosilicates ranging from 0.5% to 10% by weight relative to the total weight of the composition.

24. The composition according to claim 1, wherein the exfoliated phyllosilicates are derived from the intercalation of smectites.

25. The composition according to claim 24, wherein the smectites are chosen from the group consisting of montmorillonites, nontronites, beidellites, volkonskoites, hectorites, saponites, sauconites, sobockites, stevensites, svinfordites and vermiculites.

26. The composition according to claim 25, wherein the smectites are a mixture of at least two members chosen from the group consisting of montmorillonites, nontronites, beidellites, volkonskoites, hectorites, saponites, sauconites, sobockites, stevensites, svinfordites and vermiculites.

27. The composition according to claim 1, wherein the exfoliated phyllosilicates contain at least 15% by weight of intercalating agent relative to the weight of dry phyllosilicate.

28. The composition according to claim 27, wherein the exfoliated phyllosilicates contain at least 20% by weight of intercalating agent relative to the weight of dry phyllosilicate.

29. The composition according to claim 28, wherein the exfoliated phyllosilicates contain at least 30% by weight of intercalating agent relative to the weight of dry phyllosilicate.

30. The composition according to claim 1, wherein the intercalating agent is chosen from the group consisting of oniums containing at least one C₁ to C₅₀ hydrocarbon-based chain, polymeric organic compounds and non-polymeric organic compounds.

31. The composition according to claim 30, wherein the polymeric organic compounds contain at least one group chosen from the group consisting of aromatic rings and polar groups.

32. The composition according to claim 31, wherein the at least one group is chosen from the group consisting of carboxyl, hydroxyl, polyol, carboxylic acid, aldehyde, ketone, linear amine, cyclic amine, linear amide cyclic amide, ester, lactone and ether groups.

33. The composition according to claim 30, wherein the non-polymeric organic compounds contain at least one group chosen from the group consisting of aromatic rings and polar groups.

34. The composition according to claim 33, wherein the at least one group is chosen from the group consisting of carboxyl, hydroxyl, polyol, carboxylic acid, aldehyde, ketone, linear amine, cyclic amine, linear amide cyclic amide, ester, lactone and ether groups.

35. The composition according to claim 30, wherein the oniums are chosen from the group consisting of primary, secondary, tertiary and quaternary ammoniums containing at least one C₄-C₅₀ alkyl chain.

36. The composition according to claim 30, wherein the non-polymeric organic compound contains a polar group and at least one hydrophobic chain.

37. The composition according to claim 36, wherein the hydrophobic chain is chosen from the group consisting of a C₄ to C₅₀ alkyl, a C₄ to C₅₀ alkylene, and a C₄ to C₅₀ alkylaryl chain.

38. The composition according to claim 36, wherein the non-polymeric organic compound is an alkylpyrrolidone with a C₄ to C₅₀.

39. The composition according to claim 38, wherein the non-polymeric organic compound is an alkylpyrrolidone with a C₈ to C₃₀ alkyl chain.

40. The composition according to claim 30, wherein the polymeric organic compound is a synthetic oligomer or polymer containing at least one group chosen from the group consisting of an aromatic nucleus and a polar group.

41. The composition according to claim 40, wherein the at least one group is chosen from the group consisting of polyvinylpyrrolidone (PVP) derivatives, polyvinyl alcohol (PVA) derivatives, polyacrylic derivatives in their polymeric and copolymeric forms, polymethacrylic acid (PMAA) derivatives, polyvinyloxazolidone (PVO) derivatives, polyvinylmethyloxazolidone (PVMO) derivatives and polyvinyloxazoline derivatives.

42. The composition according to claim 1, wherein the exfoliated phyllosilicates are chosen from the group consisting of exfoliated phyllosilicates derived from montmorillonite-PVP, exfoliated phyllosilicates derived from montmorillonite-PVA and exfoliated phyllosilicates derived from montmorillonite-alkylpyrrolidone.

43. The composition according to claim 42, wherein the exfoliated phyllosilicates are a mixture of at least two members chosen from the group consisting of exfoliated phyllosilicates derived from montmorillonite-PVP, exfoliated phyllosilicates derived from montmorillonite-PVA and exfoliated phyllosilicates derived from montmorillonite-alkylpyrrolidone.

44. The composition according to claim 1, wherein the composition is in the form of an emulsion.

45. The composition according to claim 44, wherein the emulsion is a direct or inverse emulsion.

46. The composition according to claim 44, wherein the emulsion is in a form chosen from the group consisting of water-in-oil, oil-in-water and multiple emulsions.

47. The composition according to claim 44, wherein the emulsion comprises at least one emulsifying agent.

48. The composition according to claim 47, wherein the emulsion comprises at least one co-emulsifying agent.

49. The composition according to claim 48, wherein the emulsifying and co-emulsifying agents are present at a proportion of less than 30% by weight relative to the total weight of the composition.

50. The composition according to claim 49, wherein the emulsifying and co-emulsifying agents are present at a proportion of less than 20% by weight relative to the total weight of the composition.

51. The composition according to claim 50, wherein the emulsifying and co-emulsifying agents are present at a proportion of less than 15% by weight relative to the total weight of the composition.

52. The composition according to claim 51, wherein the emulsifying and co-emulsifying agents are present at a proportion of less than 0.5% by weight relative to the total weight of the composition.

53. The composition according to claim 52, wherein the emulsion is free of any emulsifying and co-emulsifying agents.

54. The composition according to claim 1, comprising a continuous fatty phase.

55. The composition according to claim 1, wherein the composition is anhydrous.

56. The composition according to claim 1, wherein the fatty phase contains at least one fatty substance chosen from the group consisting of a fatty substance that is liquid at room temperature and atmospheric pressure and a fatty substance that is solid at room temperature and atmospheric pressure.

57. The composition according to claim 56, wherein the fatty phase is a mixture of at least one fatty substance that is liquid at room temperature and atmospheric pressure and at least one fatty substance that is solid at room temperature and atmospheric pressure.

58. The composition according to claim 56, wherein the fatty substance that is liquid at room temperature and atmospheric pressure comprises at least one oil chosen from the group consisting of a volatile oil and a non-volatile oil.

59. The composition according to claim 58, wherein the fatty substance that is liquid at room temperature and atmospheric pressure is a mixture of at least one volatile oil and at least one non-volatile oil.

60. The composition according to claim 58, wherein the non-volatile oil is chosen from the group consisting of hydrocarbon-based oils of animal origin; hydrocarbon-based plant oils; linear or branched hydrocarbons of mineral or synthetic origin; synthetic ethers containing from 10 to 40 carbon atoms; synthetic esters; polyol esters; fatty alcohols that are liquid at room temperature, containing from 12 to 26 carbon atoms; higher fatty acids; and silicone oils of polymethylsiloxane (PDMS) type.

61. The composition according to claim 58, wherein the non-volatile oil is a mixture of at least two members chosen from the group consisting of hydrocarbon-based oils of animal origin; hydrocarbon-based plant oils; linear or branched hydrocarbons of mineral or synthetic origin; synthetic ethers containing from 10 to 40 carbon atoms; synthetic esters; polyol esters; fatty alcohols that are liquid at room temperature, containing from 12 to 26 carbon atoms; higher fatty acids; and silicone oils of polymethylsiloxane (PDMS) type.

62. The composition according to claim 58, wherein the volatile oil is chosen from the group consisting of hydrocarbon-based oils containing from 8 to 16 carbon atoms and volatile silicone oils.

63. The composition according to claim 56, wherein the fatty substance that is liquid at room temperature and atmospheric pressure is present in an amount of from 0.01% to 90% by weight relative to the total weight of the fatty phase.

64. The composition according to claim 63, wherein the fatty substance that is liquid at room temperature and atmospheric pressure is present in an amount of from 0.1% to 85% by weight relative to the total weight of the fatty phase.

65. The composition according to claim 56, wherein the fatty substance that is solid at room temperature and atmospheric pressure is chosen from the group consisting of waxes, pasty fatty substances and gums.

66. The composition according to claim 65, wherein the fatty substance that is solid at room temperature and atmospheric pressure is a mixture of the waxes, the pasty fatty substances and the gums.

67. The composition according to claim 56, wherein the fatty substance that is solid at room temperature and atmospheric pressure is present in an amount of from 0.01% to 50% by weight relative to the total weight of the composition.

68. The composition according to claim 67, wherein the fatty substance that is solid at room temperature and atmospheric pressure is present in an amount of from 0.1% to 40% by weight relative to the total weight of the composition.

69. The composition according to claim 68, wherein the fatty substance that is solid at room temperature and atmospheric pressure is present in an amount of from 0.2% to 30% by weight relative to the total weight of the composition.

70. The composition according to claim 1, wherein the composition also comprises a particulate phase in a proportion of from 0.01% to 40% by weight relative to the total weight of the composition.

71. The composition according to claim 70, wherein the particulate phase is present in an amount of from 0.01% to 30% by weight relative to the total weight of the composition.

72. The composition according to claim 71, wherein the particulate phase is present in an amount of from 0.05% to 20% by weight relative to the total weight of the composition.

73. The composition according to claim 70, wherein the particulate phase comprises at least one additional member chosen from the group consisting of pigments, nacres and fillers.

74. The composition according to claim 73, wherein the particulate phase is a mixture of at least two members of the group consisting of pigments, nacres and fillers.

75. The composition according to claim 1, wherein the composition comprises a film-forming polymer.

76. The composition according to claim 75, wherein the film-forming polymer is chosen from the group consisting of liposoluble film-forming polymers and lipodispersible film-forming polymers.

77. The composition according to claim 76, wherein the film-forming polymer is a mixture of the liposoluble film-forming polymers and the lipodispersible film-forming polymers.

78. The composition according to claim 1, wherein the composition is in a form of a cast product.

79. The composition according to claim 78, wherein the form of the cast product is chosen from the group consisting of a stick form and a dish form.

80. The composition according to claim 1, wherein the composition is in a form chosen from the group consisting of lipsticks, lip balms, cast foundations, concealer products, complexion “correctors”, complexion “enhancers”, eyeshadows and makeup rouges.

81. The composition according to claim 1, wherein the composition is in a form of a composition for caring for and/or making up natural or synthetic nails.

82. A cosmetic composition for caring for and/or making up skin, lips and/or integuments, in a form of an emulsion, the composition comprising a physiologically acceptable medium containing at least one fatty phase and at least an effective amount of exfoliated phyllosilicates; wherein:
the exfoliated phyllosilicates are derived from at least one phyllosilicate intercalated with one or more molecules of an intercalating agent; and
the compositions is free of any emulsifying and co-emulsifying agent.

83. A method for texturizing a fatty phase of a cosmetic composition for caring for and/or making up skin, lips and/or integuments comprising adding exfoliated phyllosilicates derived from at least one phyllosilicate intercalated with one or more molecules of a non-polyphenolic intercalating agent to the fatty phase.

84. The method according to claim 83, wherein the composition is an emulsion.

85. A method for stabilizing an emulsion comprising preparing the emulsion with exfoliated phyllosilicates derived from at least one phyllosilicate intercalated with one or more molecules of a non-polyphenolic intercalating agent.

86. A method for adjusting the staying power, migration-resistance and/or transfer-resistance properties of a cosmetic composition for making up and/or caring for skin, lips and/or integuments comprising adding exfoliated phyllosilicates derived from at least one phyllosilicate intercalated with one or more molecules of a non-polyphenolic intercalating agent to the composition.

87. A method for matting, smoothing out and/or unifying the complexion and/or for attenuating skin relief defects comprising adding exfoliated phyllosilicates derived from at least one phyllosilicate intercalated with one or more molecules of a non-polyphenolic intercalating agent to a composition for caring for and/or making up skin.

88. A process for making up skin, lips and/or integuments, comprising applying at least one layer of the composition according to claim 1 to the skin, the lips and/or the integuments.

89. A made-up synthetic support comprising the composition according to claim 1 on at least part of its surface.
